(19) United States
(12) Patent Application Publication
(10) Pub. No.: US 2024/0216050 A1
(43) Pub. Date: Jul. 4, 2024

(54) MULTI-ELECTRODE CATHETER WITH INTERLACED SUBSTRATE

(71) Applicant: Biosense Webster (Israel) Ltd., Yokneam (IL)

(72) Inventor: Juan RODRIGUEZ SOTO, Irvine, CA (US)

(21) Appl. No.: 18/508,429

(22) Filed: Nov. 14, 2023

Related U.S. Application Data
(60) Provisional application No. 63/477,673, filed on Dec. 29, 2022.

(51) Int. Cl. A61B 18/14

(52) U.S. Cl. CPC A61B 18/1492; A61B 2018/0016

(57) ABSTRACT

Catheters are presented herein having at least one electrode pair with a first electrode in the pair configured to contact tissue to receive and/or apply electrical signals for mapping and/or ablation and a second electrode in the pair being positioned facing an opposite direction, away from the tissue, and configured to function as a reference electrode for the first electrode. The first and second electrodes are positioned on separate substrates that are interwoven with each other.

### CROSS-REFERENCE TO RELATED APPLICATIONS

This application claims the benefit of prior filed U.S. Provisional Patent Application No. 63/477,673 filed on Dec. 29, 2022, which is hereby incorporated by reference as set forth in full herein.

### FIELD

The present invention relates generally to multi-electrode catheters for diagnostic and therapeutic purposes and particularly to planar multi-electrode catheters.

### BACKGROUND

Cardiac arrhythmias, such as atrial fibrillation (AF), occur when regions of cardiac tissue abnormally conduct electric signals to adjacent tissue. This disrupts the normal cardiac cycle and causes asynchronous rhythm. Certain procedures exist for treating arrhythmia, including surgically disrupting the origin of the signals causing the arrhythmia and disrupting the conducting pathway for such signals. By selectively ablating cardiac tissue by application of energy via a catheter, it is possible to cease or modify the propagation of unwanted electrical signals from one portion of the heart to another.

Many current ablation approaches in the art tend to utilize radiofrequency (RF) electrical energy to heat tissue. RF ablation can have certain rare drawbacks due to operator's skill, such as heightened risk of thermal cell injury which can lead to tissue charring, burning, steam pop, phrenic nerve palsy, pulmonary vein stenosis, and esophageal fistula. Cryoablation is an alternative approach to RF ablation that can reduce some thermal risks associated with RF ablation but may present tissue damage due to the very low temperature nature of such devices. Maneuvering cryoablation devices and selectively applying cryoablation, however, is generally more challenging compared to RF ablation; therefore, cryoablation is not viable in certain anatomical geometries which may be reached by electrical ablation devices.

Some ablation approaches use irreversible electroporation (IRE) to ablate cardiac tissue using nonthermal ablation methods. IRE delivers short pulses of high voltage to tissues and generates an unrecoverable permeabilization of cell membranes. Delivery of IRE energy to tissues using multi-electrode catheters was previously proposed in the patent literature. Examples of systems and devices configured for IRE ablation are disclosed in U.S. Patent Pub. No. 2021/0169550A1, 2021/0169567A1, 2021/0169568A1, 2021/0161592A1, 2021/0196372A1, 2021/0177503A1, and 2021/0186604A1, each of which are incorporated herein by reference and attached to the Appendix of priority patent Application No. 63/477,673.

The same or different catheter can be used to perform ablation and mapping, which respectively have different design considerations. Alternative catheter designs are therefore desired to meet various design requirements for mapping and/or ablation treatments.

### SUMMARY

Catheters are presented herein having at least one electrode pair with a first electrode in the pair configured to contact tissue to receive and/or apply electrical signals for mapping and/or ablation and a second electrode in the pair being positioned facing an opposite direction, away from the tissue, and configured to function as a reference electrode for the first electrode. The first and second electrodes are positioned on separate substrates that are interwoven with each other.

An example end effector of a catheter includes a planar substrate, an interlaced substrate, and an electrode pair. The planar substrate can extend along a longitudinal axis. The planar substrate can include openings therethrough. The interlaced substrate can traverse on a first side of the end effector across a first side of the planar substrate, extend through an opening through the planar substrate, and traverse on a second side of the end effector across a second side of the planar substrate opposite the first side of the planar substrate. The electrode pair can include an electrode affixed to the planar substrate and an electrode affixed to the interlaced substrate substantially opposite the electrode affixed to the planar substrate.

The planar substrate can further include a frame, a first membrane, and a second membrane. The frame can include one or more struts extending along the longitudinal axis. The first membrane can extend between the one or more struts on a first side of the planar substrate facing the first side of the end effector. The second membrane can extend between the one or more struts on a second side of the planar substrate facing the second side of the end effector. A portion of the first membrane can be exposed to ambient environment. A portion of the first membrane can be traversed by the interlaced substrate. A portion of the second membrane can be exposed to ambient environment. A portion of the second membrane can be traversed by the interlaced substrate.

The electrodes of the electrode pair can be exposed to ambient environment.

A first electrode of the electrode pair can be configured to contact tissue. A second electrode of the electrode pair can be configured as a reference electrode to the first electrode.

A center of a first electrode of the electrode pair can be substantially aligned with a center of a second electrode of the electrode pair.

The planar substrate can further include a frame and a membrane. The frame can include a pair of longitudinally extending struts that extend along the longitudinal axis. The membrane can extend over each strut of the pair of longitudinally extending struts and between the pair of longitudinally extending struts. The electrode affixed to the planar substrate of the electrode pair can be affixed to the membrane and between the pair of longitudinally extending struts.

The end effector can include an array of at least four electrodes on the first side of the end effector. The array can include an electrode of the electrode pair that is on the first side of the end effector. The electrodes of the array can be coplanar with each other.

Another example end effector of a catheter can include a plurality of spines, an interlaced substrate, and an electrode pair. The interlaced substrate can traverse across a first spine of the plurality of spines on a first side of the end effector and traverse across a second spine of the plurality of spines on a second side of the end effector. The electrode pair can include an electrode affixed to a spine of the plurality of spines and an electrode affixed the interlaced substrate substantially opposite the electrode affixed to the spine.

The electrodes of the electrode pair can be exposed to ambient environment.

A first electrode of the electrode pair can be configured to contact tissue. A second electrode of the electrode pair can be configured as a reference electrode to the first electrode.

A center of a first electrode of the electrode pair can be substantially aligned with a center of a second electrode of the electrode pair.

The plurality of spines can be substantially coplanar.

The end effector can include a loop member having two spines of the plurality of spines and a connecting member joining distal ends of the two spines.

The end effector can include a first loop member and a second loop member. The first loop member can include a first pair of spines of the plurality of spines and a first connecting member joining distal ends of spines of the first pair of spines. The second loop member can include a second pair of spines of the plurality of spines and a second connecting member joining distal ends of spines of the second pair of spines. The interlaced substrate can traverse each spine of the first pair of spines and the second pair of spines. The end effector can further include a first plurality of electrodes and a second plurality of electrodes. The first plurality of electrodes can include a respective electrode affixed to each spine of the first pair of spines. The second plurality of electrodes can be affixed to the interlaced substrate and respectively opposite at least a portion of the first plurality of electrodes.

Another end effector of a catheter can include a top side, a bottom side, a first substrate portion, a second substrate portion, an opening between the first and second substrate portions, an interlaced substrate, a first electrode pair, and a second electrode pair. The bottom side is opposite the top side. The interlaced substrate can traverse on the top side of the end effector across the first substrate portion, extend through the opening, and traverse on a bottom side of the end effector across the second substrate portion. The first electrode pair can have a bottom electrode affixed to the first substrate portion on the bottom side of the end effector and a top electrode affixed to the interlaced substrate on the top side of the end effector. The top electrode can be disposed substantially opposite the bottom electrode of the first electrode pair. The second electrode pair can include a top electrode affixed to the second substrate portion on the top side of the end effector and a bottom electrode affixed to the interlaced substrate on the bottom side of the end effector. The bottom electrode can be disposed substantially opposite the top electrode of the second electrode pair.

Electrodes of the first electrode pair and the second electrode pair each can be exposed to ambient environment.

The top electrodes of the first electrode pair and the second electrode pair can be configured to contact tissue. The bottom electrodes of the first electrode pair and the second electrode pair can be configured as reference electrodes for the top electrode in the respective electrode pair.

The interlaced substrate can be a first interlaced substrate, and the end effector can further include a second interlaced substrate extending through the opening and traversing on the top side of the end effector and the bottom side of the end effector.

The first substrate portion, the second substrate portion, and the opening can each be elongated along a longitudinal axis. The interlaced substrate can extend orthogonal to the longitudinal axis.

The interlaced substrate can include a flex circuit.

The end effector can include an array of at least four electrodes on the top side of the end effector. The array can include the top electrodes of the first electrode pair and the second electrode pair. The electrodes of the array can be coplanar with each other.

The end effector can further include a support frame configured to move the first substrate portion, the second substrate portion, and the interlaced substrate into a predetermined shape. The predetermined shape being planar. Alternatively, the predetermined shape can be selected from a group including a spheroid, conic or truncated cone.

The support frame can include a plurality of longitudinally extending struts.

The end effector can further include a contiguous substrate including the first substrate portion and the second substrate portion. The contiguous substrate can further include a first membrane and a second membrane. The first membrane can face the top side of the end effector such that a portion of the first membrane is exposed to ambient environment and a portion of the first membrane is traversed by the interlaced substrate. The second membrane can face the bottom side of the end effector such that a portion of the second membrane is exposed to ambient environment and a portion of the second membrane is traversed by the interlaced substrate. The contiguous substrate can further include a nitinol support frame extending through the first substrate portion and/or the second substrate portion. The frame can have longitudinally extending struts, and at least one of the membrane portions can be disposed between the longitudinally extending struts.

The end effector can further include a support frame assembly having a first longitudinally extending strut and a second longitudinally extending strut such that the first substrate portion includes the first longitudinally extending strut and the second substrate portion includes the second longitudinally extending strut. The first longitudinally extending strut can be disposed between electrodes of the first electrode pair. The second longitudinally extending strut can be disposed between a electrodes of the second electrode pair.

The end effector can further include a third substrate portion, a fourth substrate portion, and respective openings between the second and third substrate portions and the third and fourth substrate portions. The interlaced substrate can traverse the third substrate portion and the fourth substrate portion. The interlaced substrate can extend through the respective openings and traverse on the bottom side of the end effector across the third substrate portion and traverse on the top side of the end effector across the fourth substrate portion.

The support frame assembly can further include a third longitudinally extending strut and a fourth longitudinally extending strut such that the third substrate portion includes the third longitudinally extending strut and the fourth substrate portion includes the fourth longitudinally extending strut. The support frame assembly can further include a first connecting strut and a second connecting strut such that the first connecting strut joins the first longitudinally extending strut and the third longitudinally extending strut, and the second connecting strut joins the second longitudinally extending strut and the fourth longitudinally extending strut. The first connecting strut can be arcuate, and the second connecting strut can be arcuate.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of a first example end effector of a catheter according to aspects of the present invention.

FIG. 2 is a cross-sectional illustration of the first example end effector as indicated in FIG. 1.

FIG. 3 is an illustration of a second example end effector of a catheter according to aspects of the present invention.

FIG. 4 is a cross-sectional illustration of the second example end effector as indicated in FIG. 3.

FIG. 5 is an illustration of a third example end effector of a catheter according to aspects of the present invention.

FIG. 6 is an illustration of an example catheter-based electrophysiology mapping and ablation system according to aspects of the present invention.

### DETAILED DESCRIPTION

The following detailed description should be read with reference to the drawings, which depict selected embodiments and are not intended to limit the scope of the invention. This description enables one skilled in the art to make and use the invention, and describes several embodiments, adaptations, variations, alternatives and uses of the invention, including what is presently believed to be the best mode of carrying out the invention. Any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the pertinent art in view of the teachings herein. Such modifications and variations are intended to be included within the scope of the claims.

As used herein, the terms “patient,” “host.” “user,” and “subject” refer to any human or animal subject and are not intended to limit the systems or methods to human use, although use of the subject invention in a human patient represents a preferred embodiment. As well, the term “proximal” indicates a location closer to the operator whereas “distal” indicates a location further away to the operator or physician.

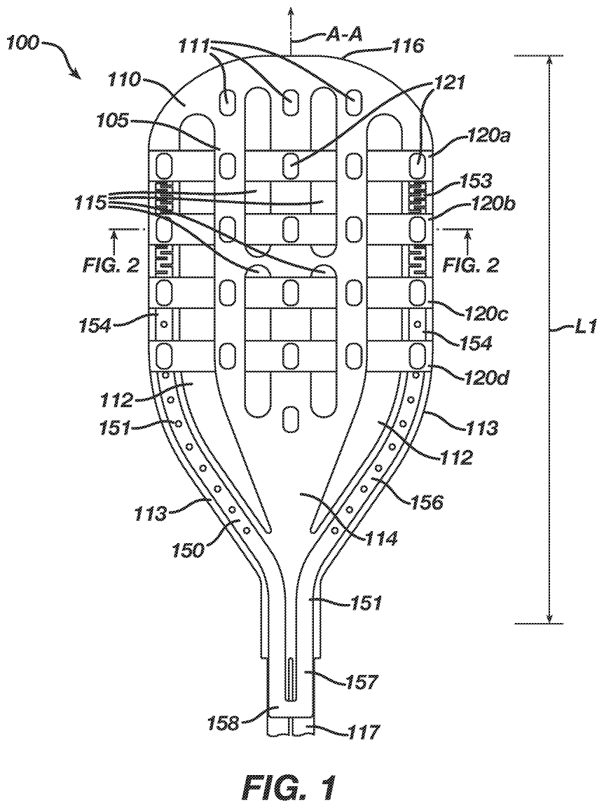

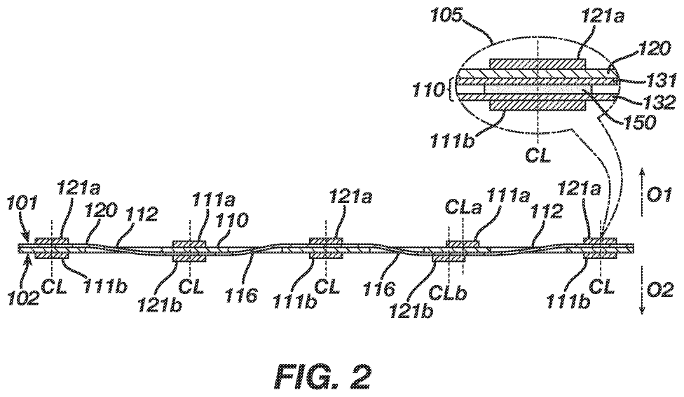

FIG. 1 is an illustration of a first example end effector 100 of a catheter. The end effector 100 includes a planar substrate 110 and multiple interlaced substrates 120a. 120b, 120c, 120d that go over and under portions of the planar substrate 110 at overlapping regions 105. The end effector 100 includes electrodes 111 affixed to the planar substrate and electrodes 121 affixed to the interlaced substrates 120a, 120b, 120c, 120d. A first side 101 (FIG. 2) of the end effector 100 is illustrated in FIG. 1. The first side 101 includes an array of electrodes 111, 121 that are coplanar with each other. The end effector 100 can have a second side 102 (FIG. 2) opposite the first side with one or more electrodes such that at least one electrode on the second side is opposite an electrode on the first side, forming an electrode pair with the opposite electrode.

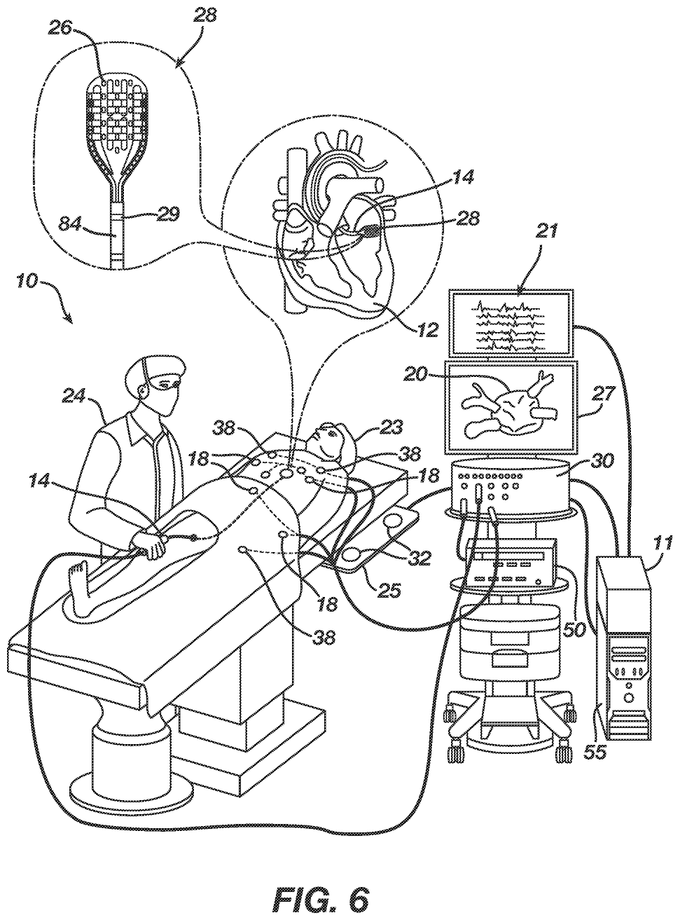

The planar substrate 110 extends along a longitudinal axis A-A and includes openings 112, 115 through the planar substrate 110. The interlaced substrates 120a, 120b, 120c, 120d extend through the openings 112, 115 to interlace with the planar substrate 110. The openings include two outer longitudinal openings 112 that extend along the longitudinal axis A-A and through a majority of a length L1 of the end effector 100. The two outer longitudinal openings 112 divide the planar substrate 110 into outer portions 113 and a central portion 114. The openings include central longitudinal openings 115 that are between the outer longitudinal openings 112 and extend along the longitudinal axis A-A within the central portion 114 of the planar substrate 110. The planar substrate 110 includes a distal arc 116 at a distal end of the end effector 100. The planar substrate 110 includes a proximal extension 117 that is configured to be coupled to a catheter shaft to form a catheter 14 (FIG. 6).

The planar substrate 110 further includes a support frame 150 having a proximal connecting portion 158 joining two distally extending struts 151. The struts 151 include proximal strut portions 157 extending distally from the proximal connecting portion 158 and along the longitudinal axis A-A, angled strut portions 156 extending distally from the proximal strut portions 157 and angling away from the longitudinal axis A-A, and outer strut portions 154 extending parallel to each other along the longitudinal axis A-A distal of the angled strut portions 156 and including a graduated cut pattern 153. At least some of the electrodes 121 positioned in the outer portions 113 of the planar substrate 110 are positioned over the outer strut portions 154. A majority of electrodes 121, 111 are positioned between the outer strut portions 154.

The interlaced substrates 120a, 120b, 120c, 120d are manufactured as separate from the planar substrate 110 and are coupled to the planar substrate 110 with a coupling mechanism, which can include adhesive, weld, rivet, or other suitable coupling means as understood by a person skilled in the pertinent art. The interlaced substrates 120a, 120b, 120c, 120d are affixed to the outer portions 113 of the planar substrate 110 near ends of the interlaced substrates 120a, 120b, 120c, 120d. The interlaced substrates 120a, 120b, 120c, 120d may also be coupled to the central portions 114 of the planar substrate 110.

The interlaced substrates 120a, 120b, 120c, 120d are perpendicular to the longitudinal axis A-A. The interlaced substrates are woven over and under portions of the planar substrate 110 in an alternating pattern with a fixed distance between interlaced substrates. Alternatively, the interlaced substrates 120a, 120b, 120c, 120d need not be interlaced and can each be entirely on one side of the end effector 200. As another alternative, the interlaced substrates 120a, 120b, 120c, 120d need not alternate between the sides of the end effector through every available opening 112. 115, but may cross on the same side of the end effector 100 on two or more adjacent portions of the planar substrate 110, then cross through an opening 112, 115 to the opposite side of the end effector 100 to traverse another portion of the planar substrate 110.

FIG. 2 is a cross-sectional illustration of the first example end effector 100 as indicated in FIG. 1. The end effector 100 has a first side 101 facing up and a second side 102 facing down as illustrated in FIG. 2. From left to right, the interlaced substrate 120 is positioned on the first side 101 of the end effector 100 across a first side of the planar substrate 110, extends through an outer longitudinal opening 112 of the planar substrate 110, traverses on the second side 102 of the end effector 100 across a second side of the planar substrate opposite the first side of the planar substrate, extends through a central opening 116 of the planar substrate 110, traverses on the first side 101 of the end effector 100 across the first side of the planar substrate 110, extends through another central opening 116 of the planar substrate 110, traverses on the second side 102 of the end effector 100 across the second side of the planar substrate 110, extends through another outer longitudinal opening 112 of the planar substrate 110, and traverses on the first side 101 of the end effector 100 across the first side of the planar substrate 110.

The end effector 100 includes electrode pairs each having an electrode affixed to the planar substrate 110 and an electrode affixed to the interlaced substrate 120 substantially opposite the electrode affixed to the planar substrate 110. Three of the electrode pairs (right, left and center) include an electrode 121a affixed to the interlaced substrate 120 on the first side 101 of the end effector 100 and an electrode 111b affixed to the planar substrate 110 on the second side 102 of the end effector 100. Two of the electrode pairs (between the aforementioned three electrode pairs) include an electrode 111a affixed to the planar substrate 110 on the first side 101 of the end effector 100 and an electrode 111b affixed to the interlaced substrate 120 on the second side 102 of the end effector 100. The electrodes of the electrode pairs are substantially aligned with respect to a center line CL. The centerlines CLa, CLb of the electrodes 111a, 121b of the fourth electrode pair from the left are not precisely aligned, and electrodes 111a, 121b in the fourth electrode pair are substantially opposite each other by virtue of having significant overlap within respect to the plane of the planar substrate 110.

Electrodes 111, 121, 131 in the electrode pairs are exposed to ambient environment so that when inserted in a bodily lumen during a treatment, some of the electrodes can be in contact with tissue while other electrodes are in contact with bodily fluid. By being positioned opposite each other, when one electrode in the pair is in contact with tissue, the other electrode can be configured as a reference electrode for the electrode in contact with tissue. The reference electrode can measure an electrical potential of the bodily fluid, which can be compared to an electrical potential of the opposite electrode in the pair that is in contact with tissue.

The electrodes 111, 121, 131 can be configured to function with a system similar to the system 10 illustrated in FIG. 6. The electrodes can be configured for tracking and position orientation of the end effector 100, sensing Intracardiac Electrogram (IEGM) signals, ablating using radiofrequency energy, ablating using irreversible electroporation, or any combination thereof.

The planar substrate 110 further includes a first membrane 131 on the first side of the planar substrate 110 and a second membrane 132 on the second side of the planar substrate 110. The membranes 131, 132 extends between the struts 151 of the frame 150. The first and fifth electrode pairs are over the support frame 150, and the second, third, and fourth electrode pairs are between the struts 151 of the support frame 150. Portions of the membranes 131, 132 are exposed to ambient environment, and portions of the membranes 131, 132 are traversed by the interlacing substrate 120. The membranes 131, 132 can each respectively include a flex circuit that can be configured with conductors, sensors, and/or other compatible features as understood by a person skilled in the art.

For instance, the planar substrate 110 can include features of example end effectors illustrated in FIGS. 1A-G, 2A-E, 3, and 4A-C of U.S. Provisional Patent Application No. 63/403,589 filed Sep. 2, 2022, incorporated herein by reference, a copy of which is attached to the Appendix of priority patent Application No. 63/477,673. The planar substrate 110 can include features of example end effectors illustrated in U.S. patent application Ser. No. 18/230,934 filed Aug. 7, 2023, which is incorporated by reference herein and claims priority to U.S. 63/403,589. As non-limiting examples, the planar substrate 110 can include electrically conductive traces to make contact to the electrodes 111 and sensors, piezoelectric transducers, planar spiral single axis position sensors, impedance navigation sensor, etc.

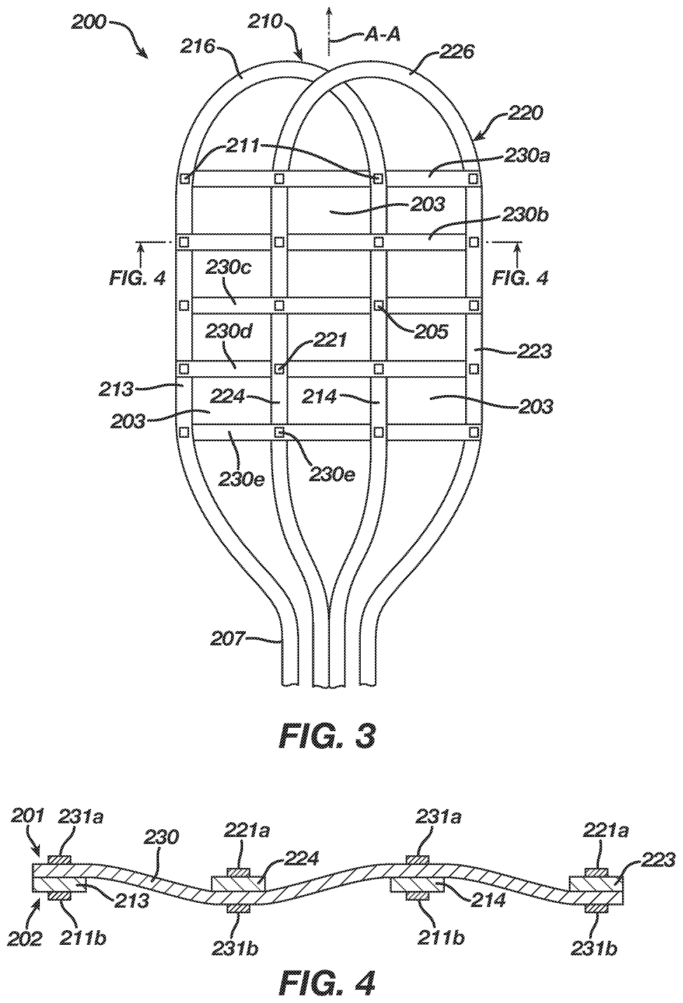

FIG. 3 is an illustration of a second example end effector 200 of a catheter including a first loop 210, a second loop 220, interlaced substrates 230a, 230b, 230c, 230d, 230c, and electrodes 211, 221, 231. The interlaced substrates 230a, 230b, 230c, 230d, 230e weave over and under spines 213, 214, 223, 224 of the loops 210, 220. The first loop 210 has a first spine 213 that is on an outer edge of the end effector 200 and a second spine 214 that is positioned centrally within the end effector 200. The second loop 220 has a first spine 223 that is on an outer edge of the end effector 200 and a second spine 224 that is positioned centrally within the end effector 200. The spines 213, 214, 223, 224 are substantially coplanar and aligned along a longitudinal axis A-A. The second spine 214, 224 of each of the first loop 210 and the second loop is positioned between spines of the other loop. The loop members 210, 220 each respectively include a connecting member 216, 226 in the shape of an arc at a distal end of the end effector 200 joining distal ends of the spines 213, 214, 223, 224 of the respective loop members. A first loop member 210 includes a first pair of spines 213, 214 and a first connecting member 216 joining distal ends of the spines 213, 214 of the first pair of spines. A second loop member 220 includes a second pair of spines 223, 224 and a second connecting member 226 joining distal ends of spines 223, 224 of the second pair of spines. A proximal portion 207 of the end effector includes ends of the loop members 210 that are configured to attach to a catheter shaft.

The end effector 200 can include an alternative number of loop members, and preferably includes two or three loop members. The loop members 210, 220 and catheter shaft can include compatible features of catheters having an end effector with loop members as understood by a person skilled in the pertinent art including catheters disclosed in U.S. Patent Publication Nos. 2021/0369339A1 and 2021/0369132A1 incorporated herein by reference, a copy of each of which is attached to the Appendix of priority patent Application No. 63/477,673. For instance, the loop members 210, 220 can include a support frame including nitinol or other suitable material to provide structural support for the loop members, electrical conductors (e.g. wiring, flex circuit traces, etc.) to connect to electrodes 211, 221, irrigation conduits, and the like. The end effector 200 can include an alternative number of loop members, for instance three loop members. The loop members 210, 220 can have an alternative shape as understood by a person skilled in the pertinent art. Alternatively, the end effector 200 can lack one or both connecting members 216, 226 so that the spines 213, 214, 223, 224 have free distal ends such as disclosed in U.S. Pat. No. 9,820,664 incorporated herein by reference, a copy of which is attached to the Appendix of priority patent Application No. 63/477,673.

The interlaced substrates 230a, 230b, 230c, 230d, 230e are perpendicular to the spines 213, 214, 223, 224 and the longitudinal axis A-A. The interlaced substrates 230a, 230b, 230c, 230d, 230e traverse each spine of the first pair of spines 213, 214 and the second pair of spines 223, 224. The spines and interlaced substrates are woven over and under each other in an alternating pattern with a fixed distance between spines and interlaced substrates similar to a tennis racket. The spines 213, 214, 223, 224 are positioned with an opening 203 between adjacent spines so that the interlaced substrates 230a, 230b, 230c, 230d, 230e can traverse across a spine one side of the end effector 200, through the opening 203, and across an adjacent spine on the opposite side of the end effector 200. Alternatively, the substrates 230a, 230b, 230c, 230d, 230e need not be interlaced and can each be entirely on one side of the end effector 200. As another alternative, the interlaced substrates 230a, 230b, 230c, 230d, 230e need not alternate between the sides of the end effector between every spine, but may cross on the same side of the end effector on two or more adjacent spines, then cross to the opposite of the end effector to traverse another spine.

The interlaced substrates 230a, 230b, 230c, 230d, 230e are manufactured separately from the loop members 210, 220 and are coupled to the loop members 210, 220 with a coupling mechanism, which may include adhesive, weld, rivet, or other suitable coupling means as understood by a person skilled in the pertinent art. Ends of the interlaced substrates 230a, 230b, 230c, 230d, 230e are affixed to the first spines 213, 223. Preferably, the interlaced substrates 230a, 230b, 230c, 230d, 230e are also affixed to the second spines 214, 224; however, they need not be.

FIG. 4 is a cross-sectional illustration of the second example end effector as indicated in FIG. 3. The interlaced substrate 230 is shown traversing across the spines 213, 224, 214, 223 on one side of the end effector, then the other in an alternating fashion. The spines 213, 224, 214, 223 and the interlaced substrate 230 include additional features not illustrated in cross section, such as conductors to electrodes and other structures of loop members 210, 220 as disclosed above. These features are omitted in FIG. 4 purely for the sake of simplicity in the illustration.

At least one, potentially all of the electrodes 211, 221, 231 of the end effector 200 form a respective electrode pair that includes an electrode 211, 221 affixed to a spine 213, 214, 223, 224 and an electrode 231 affixed to an interlaced substrate 230a, 230b, 230c, 230d, 230e. As illustrated in FIG. 4, a first electrode pair includes an electrode 231a on a first side 201 of the end effector 200 that is affixed to the interlaced substrate 230 and an opposite electrode 211b on a second side 202 of the end effector 200 that is affixed to the first spine 213 of the first loop member 210. A second electrode pair includes an electrode 221a on the first side 201 of the end effector 200 that is affixed to the second spine 224 of the second loop member 220 and an opposite electrode 231b on the second side 202 of the end effector 200 that is affixed to the interlaced substrate 230. A third electrode pair includes an electrode 231a on the first side 201 of the end effector 200 that is affixed to the interlaced substrate 230 and an opposite electrode 211b on the second side 202 of the end effector 200 that is affixed to the second spine 214 of the first loop member 210. Some electrodes 221a on the first side 201 of the end effector 200 are therefore affixed to spines 223, 224 of the second loop member 200, and some electrodes 231a on the first side 201 of the end effector 200 are therefore affixed to the interlaced substrate 230. Some electrodes 211b on the second side 202 of the end effector 200 are affixed to spines 213, 214 of the first loop member 210 and some electrodes 231b on the second side 202 of the end effector 200 are affixed to the interlaced substrate 230. Similar to as illustrated in FIG. 2, the opposite electrodes can overlap with respect to a plane of the end effector 200. The opposite electrodes can be aligned with respect to a centerline CL, or the centerlines CLa, CLb of the opposite electrodes can be offset. Electrodes in an electrode pair can be substantially opposite each other by virtue of having significant overlap within respect to a plane of the end effector 200.

Electrodes 211, 221, 231 in the electrode pairs are exposed to ambient environment so that when inserted in a bodily lumen during a treatment, a portion of the electrodes can be in contact with tissue while another portion is in contact with bodily fluid. By being positioned opposite each other, when one electrode in the pair is in contact with tissue, the other electrode can be configured as a reference electrode for the electrode in contact with tissue. The reference electrode can measure an electrical potential of the bodily fluid, which can be compared to an electrical potential of the opposite electrode in the pair that is in contact with tissue.

The electrodes 211, 221, 231 can be configured to function with a system similar to the system 10 illustrated in FIG. 6. The electrodes can be configured for tracking and position orientation of the end effector 100, sensing Intracardiac Electrogram (IEGM) signals, ablating using radiofrequency energy, ablating using irreversible electroporation, or any combination thereof.

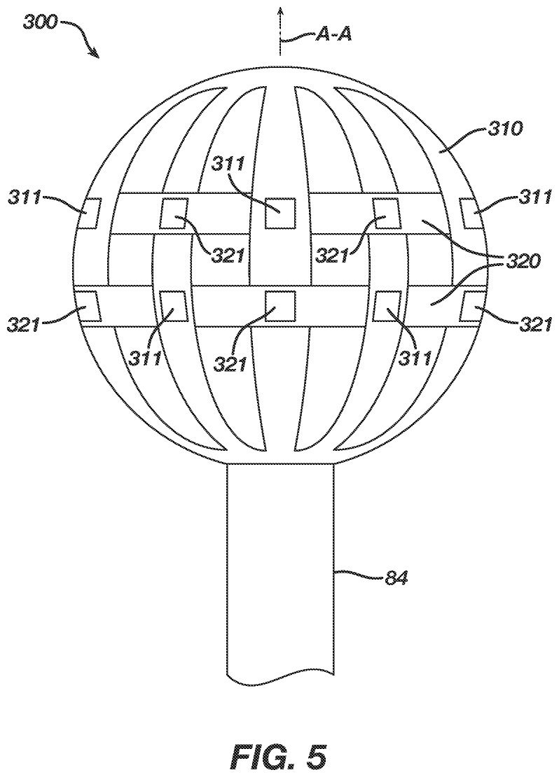

FIG. 5 is an illustration of a third example end effector 300 of a catheter coupled to a shaft 84. The end effector 300 includes a spherical substrate 310 and multiple interlaced substrates 320 that go over and under portions of the spherical substrate 320. The end effector 300 includes electrodes 311 affixed to the spherical substrate 310 and electrodes 321 affixed to the interlaced substrates 320. The illustrated electrodes 311, 321 are on an outer surface of the end effector 300 and therefore are positioned to contact tissue. The end effector 300 can further include one or more electrodes on an inner surface of the end effector 300 that are configured as act as a reference electrode to one or more of the electrodes 311, 321 on the outer surface. At least one electrode on the inner surface can be opposite an electrode on the outer surface, forming an electrode pair with the opposite electrode. The electrode pair can be configured similarly to the electrode pairs disclosed in relation to FIGS. 1 through 4.

The spherical substrate 310 extends along a longitudinal axis A-A and includes openings through the spherical substrate 310. The interlaced substrates 320 extend through the openings to interlace with the spherical substrate 310. The openings can extend longitudinally while the interlaced substrates 320 extend orthogonal to the longitudinal axis A-A. The spherical substrate 310 can further include a support frame.

The third example end effector 300 can include a flex circuit and other features similar to the first example end effector 100, reconfigured in a spherical shape. Likewise, features of the first example end effector 100 can be reconfigured into a cylindrical shape as understood by a person skilled in the pertinent art. Further, the second example end effector 200 can be adapted into a spherical or cylindrical shape as understood by a person skilled in the pertinent art.

FIG. 6 is an illustration showing an example catheter-based electrophysiology mapping and ablation system 10. The system 10 includes multiple catheters, which are percutaneously inserted by a physician 24 through the patient's vascular system into a chamber or vascular structure of a heart 12. Typically, a delivery sheath catheter is inserted into the left or right atrium near a desired location in the heart 12. Thereafter, a plurality of catheters can be inserted into the delivery sheath catheter so as to arrive at the desired location. The plurality of catheters may include catheters dedicated for sensing Intracardiac Electrogram (IEGM) signals, catheters dedicated for ablating and/or catheters dedicated for both sensing and ablating. An example catheter 14 that is configured for sensing IEGM and/or ablation is illustrated herein. The physician 24 brings a distal tip 28 of the catheter 14 into contact with the heart wall for sensing a target site in the heart 12. The distal tip 28 of the catheter can include an end effector configured similarly to the example end effectors 100, 200, 300 disclosed herein, variations thereof, and alternatives thereto as understood by a person skilled in the art. The illustrated catheter 14 include electrodes 26 that can be configured similarly to electrodes 111, 121, 211, 221, 311, 321 of the example end effectors 100, 200, 300 illustrated in FIGS. 1 through 5.

The catheter 14 may additionally include a position sensor 29 embedded in or near distal tip 28 (e.g. in shaft 84) for tracking position and orientation of distal tip 28. Optionally and preferably, position sensor 29 is a magnetic based position sensor including three magnetic coils for sensing three-dimensional (3D) position and orientation.

A magnetic based position sensor 29 may be operated together with a location pad 25 including a plurality of magnetic coils 32 configured to generate magnetic fields in a predefined working volume. Real time position of a distal tip 28 of the catheter 14 may be tracked based on magnetic fields generated with a location pad 25 and sensed by a magnetic based position sensor 29. Details of the magnetic based position sensing technology are described in U.S. Pat. Nos. 5,391,199; 5,443,489; 5,558,091; 6,172,499; 6,239,724; 6,332,089; 6,484,118; 6,618,612; 6,690,963; 6,788,967; 6,892,091 incorporated by reference herein.

The system 10 includes one or more electrode patches 38 positioned for skin contact on the patient 23 to establish location reference for location pad 25 as well as impedance-based tracking of electrodes 26. For impedance-based tracking, electrical current is directed toward electrodes 26 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182 incorporated by reference herein.

A recorder 11 displays electrograms 21 captured with body surface ECG electrodes 18 and intracardiac electrograms (IEGM) captured with electrodes 26 of the catheter 14. The recorder 11 may include pacing capability for pacing the heart rhythm and/or may be electrically connected to a standalone pacer.

The system 10 can include an ablation energy generator 50 that is adapted to conduct ablative energy to one or more of electrodes at a distal tip of a catheter configured for ablating. Energy produced by the ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

A patient interface unit (PIU) 30 is an interface configured to establish electrical communication between catheters, electrophysiological equipment, power supply and a workstation 55 for controlling operation of system 10. Electrophysiological equipment of the system 10 may include for example, multiple catheters, a location pad 25, body surface ECG electrodes 18, electrode patches 38, an ablation energy generator 50, and a recorder 11. Optionally and preferably, the PIU 30 includes processing capability for implementing real-time computations of location of the catheters and for performing ECG calculations.

The workstation 55 includes memory, processor unit with memory or storage with appropriate operating software loaded therein, and user interface capability. The workstation 55 can be configured to provide multiple functions, optionally including (1) modeling the endocardial anatomy in three-dimensions (3D) and rendering the model or an anatomical map 20 for display on a display device 27; (2) displaying on the display device 27 activation sequences (or other data) compiled from recorded electrograms 21 in representative visual indicia or imagery superimposed on the rendered anatomical map 20; (3) displaying real-time location and orientation of multiple catheters within the heart chamber; and (4) displaying on the display device 27 sites of interest such as places where ablation energy has been applied. One commercial product embodying elements of the system 10 is available as the CARTO™ 3 System, available from Biosense Webster, Inc., 31 Technology Drive, Irvine, CA 92618.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

The following clauses list non-limiting embodiments of the disclosure:

Clause 1. An end effector of a catheter, the end effector comprising: a planar substrate extending along a longitudinal axis, the planar substrate comprising openings therethrough; and an interlaced substrate traversing on a first side of the end effector across a first side of the planar substrate, extending through an opening through the planar substrate, and traversing on a second side of the end effector across a second side of the planar substrate opposite the first side of the planar substrate; and an electrode pair comprising an electrode affixed to the planar substrate and an electrode affixed to the interlaced substrate substantially opposite the electrode affixed to the planar substrate.

Clause 2. The end effector of clause 1, the planar substrate further comprising: a frame comprising one or more struts extending along the longitudinal axis; a first membrane extending between the one or more struts on a first side of the planar substrate facing the first side of the end effector; and a second membrane extending between the one or more struts on a second side of the planar substrate facing the second side of the end effector.

Clause 3. The end effector of clause 2, a portion of the first membrane being exposed to ambient environment, a portion of the first membrane being traversed by the interlaced substrate, a portion of the second membrane being exposed to ambient environment, and a portion of the second membrane being traversed by the interlaced substrate.

Clause 4. The end effector of any one of clauses 1-3, the electrodes of the electrode pair being exposed to ambient environment.

Clause 5. The end effector of any one of clauses 1-4, a first electrode of the electrode pair being configured to contact tissue, and a second electrode of the electrode pair being configured as a reference electrode to the first electrode.

Clause 6. The end effector of any one of clauses 1-5, in which a center of a first electrode of the electrode pair being substantially aligned with a center of a second electrode of the electrode pair.

Clause 7. The end effector of any one of clauses 1-6, the planar substrate further comprising: a frame comprising a pair of longitudinally extending struts that extend along the longitudinal axis; and a membrane extending over each strut of the pair of longitudinally extending struts, and between the pair of longitudinally extending struts.

Clause 8. The end effector of clause 7, the electrode affixed to the planar substrate of the electrode pair being affixed to the membrane and between the pair of longitudinally extending struts.

Clause 9. The end effector of any one of clauses 1-8, the end effector comprising an array of at least four electrodes on the first side of the end effector, the array comprising an electrode of the electrode pair that is on the first side of the end effector, and the electrodes of the array being coplanar with each other.

Clause 10. An end effector of a catheter, the end effector comprising: a plurality of spines; an interlaced substrate traversing across a first spine of the plurality of spines on a first side of the end effector and traversing across a second spine of the plurality of spines on a second side of the end effector; and an electrode pair comprising an electrode affixed to a spine of the plurality of spines and an electrode affixed the interlaced substrate substantially opposite the electrode affixed to the spine.

Clause 11. The end effector of clause 10, the electrodes of the electrode pair being exposed to ambient environment.

Clause 12. The end effector of clause 10 or 11, a first electrode of the electrode pair being configured to contact tissue, and a second electrode of the electrode pair being configured as a reference electrode to the first electrode.

Clause 13. The end effector of any one of clauses 10-12, in which a center of a first electrode of the electrode pair being substantially aligned with a center of a second electrode of the electrode pair.

Clause 14. The end effector of any one of clauses 10-13, the plurality of spines being substantially coplanar.

Clause 15. The end effector of any one of clauses 10-14, the end effector comprising: a loop member comprising two spines of the plurality of spines and a connecting member joining distal ends of the two spines.

Clause 16. The end effector of any one of clauses 10-15, the end effector comprising: a first loop member comprising a first pair of spines of the plurality of spines and a first connecting member joining distal ends of spines of the first pair of spines; and a second loop member comprising a second pair of spines of the plurality of spines and a second connecting member joining distal ends of spines of the second pair of spines.

Clause 17. The end effector of clause 16, the interlaced substrate traversing each spine of the first pair of spines and the second pair of spines.

Clause 18. The end effector of clause 16 or 17, further comprising: a first plurality of electrodes comprising a respective electrode affixed to each spine of the first pair of spines; and a second plurality of electrodes affixed to the interlaced substrate and respectively opposite at least a portion of the first plurality of electrodes.

Clause 19. An end effector of a catheter, the end effector comprising: a top side; a bottom side opposite the top side; a first substrate portion; a second substrate portion; an opening between the first and second substrate portions; an interlaced substrate traversing on the top side of the end effector across the first substrate portion, extending through the opening, and traversing on a bottom side of the end effector across the second substrate portion; a first electrode pair comprising a bottom electrode affixed to the first substrate portion on the bottom side of the end effector and a top electrode affixed to the interlaced substrate on the top side of the end effector, the top electrode being disposed substantially opposite the bottom electrode of the first electrode pair; and a second electrode pair comprising a top electrode affixed to the second substrate portion on the top side of the end effector and a bottom electrode affixed to the interlaced substrate on the bottom side of the end effector, the bottom electrode being disposed substantially opposite the top electrode of the second electrode pair.

Clause 20. The end effector of clause 19, electrodes of the first electrode pair and the second electrode pair each being exposed to ambient environment.

Clause 21. The end effector of clause 19 or 20, the top electrodes of the first electrode pair and the second electrode pair being configured to contact tissue, and the bottom electrodes of the first electrode pair and the second electrode pair being configured as reference electrodes for the top electrode in the respective electrode pair.

Clause 22. The end effector of any one of clauses 19-21, the interlaced substrate being a first interlaced substrate, and the end effector further comprising: a second interlaced substrate extending through the opening and traversing on the top side of the end effector and the bottom side of the end effector.

Clause 23. The end effector of any one of clauses 19-22, the first substrate portion, the second substrate portion, and the opening each being elongated along a longitudinal axis, and the interlaced substrate extending orthogonal to a longitudinal axis.

Clause 24. The end effector of any one of clauses 19-23, the interlaced substrate comprising a flex circuit.

Clause 25. The end effector of any one of clauses 19-24, the end effector comprising an array of at least four electrodes on the top side of the end effector, the array comprising the top electrodes of the first electrode pair and the second electrode pair, and the electrodes of the array being coplanar with each other.

Clause 26. The end effector of any one of clauses 19-25, further comprising: a support frame configured to move the first substrate portion, the second substrate portion, and the interlaced substrate into a predetermined shape.

Clause 27. The end effector of clause 26, the predetermined shape being planar.

Clause 28. The end effector of clause 26, the predetermined shape being selected from a group consisting essentially of a spheroid, conic or truncated cone.

Clause 29. The end effector of clause 26 or 27, the support frame comprising a plurality of longitudinally extending struts.

Clause 30. The end effector of any one of clauses 19-29, further comprising: a contiguous substrate comprising the first substrate portion and the second substrate portion.

Clause 31. The end effector of clause 30, the contiguous substrate further comprising: a first membrane facing the top side of the end effector such that a portion of the first membrane is exposed to ambient environment and a portion of the first membrane is traversed by the interlaced substrate; and a second membrane facing the bottom side of the end effector such that a portion of the second membrane is exposed to ambient environment and a portion of the second membrane is traversed by the interlaced substrate.

Clause 32. The end effector of clause 31, the contiguous substrate further comprising a nitinol support frame extending through the first substrate portion and/or the second substrate portion.

Clause 33. The end effector of clause 32, the frame comprising longitudinally extending struts, and the first membrane portion and/or the second membrane portion being disposed between the longitudinally extending struts.

Clause 34. The end effector of any one of clauses 19-29, further comprising: a support frame assembly comprising a first longitudinally extending strut and a second longitudinally extending strut such that the first substrate portion comprises the first longitudinally extending strut and the second substrate portion comprises the second longitudinally extending strut.

Clause 35. The end effector of clause 34, the first longitudinally extending strut being disposed between electrodes of the first electrode pair, and the second longitudinally extending strut being disposed between a electrodes of the second electrode pair.

Clause 36. The end effector of clause 34 or 35, further comprising: a third substrate portion; a fourth substrate portion; and respective openings between the second and third substrate portions and the third and fourth substrate portions, the interlaced substrate traversing the third substrate portion and the fourth substrate portion.

Clause 37. The end effector of clause 36, the interlaced substrate extending through the respective openings and traversing on the bottom side of the end effector across the third substrate portion and traversing on the top side of the end effector across the fourth substrate portion.

Clause 38. The end effector of clause 36 or 37, the support frame assembly further comprising a third longitudinally extending strut and a fourth longitudinally extending strut such that the third substrate portion comprises the third longitudinally extending strut and the fourth substrate portion comprises the fourth longitudinally extending strut.

Clause 39. The end effector of clause 38, the support frame assembly further comprising a first connecting strut and a second connecting strut such that the first connecting strut joins the first longitudinally extending strut and the third longitudinally extending strut and the second connecting strut joins the second longitudinally extending strut and the fourth longitudinally extending strut.

Clause 40. The end effector of clause 39, the first connecting strut being arcuate, and the second connecting strut being arcuate.

What is claimed is:

1. An end effector of a catheter, the end effector comprising:
a planar substrate extending along a longitudinal axis, the planar substrate comprising openings therethrough; and
an interlaced substrate traversing on a first side of the end effector across a first side of the planar substrate, extending through an opening through the planar substrate, and traversing on a second side of the end effector across a second side of the planar substrate opposite the first side of the planar substrate; and
an electrode pair comprising an electrode affixed to the planar substrate and an electrode affixed to the interlaced substrate substantially opposite the electrode affixed to the planar substrate.

2. The end effector of claim 1, the planar substrate further comprising:
a frame comprising one or more struts extending along the longitudinal axis;
a first membrane extending between the one or more struts on a first side of the planar substrate facing the first side of the end effector; and
a second membrane extending between the one or more struts on a second side of the planar substrate facing the second side of the end effector.

3. The end effector of claim 2,
a portion of the first membrane being exposed to ambient environment,
a portion of the first membrane being traversed by the interlaced substrate,
a portion of the second membrane being exposed to ambient environment, and
a portion of the second membrane being traversed by the interlaced substrate.

4. The end effector of claim 1, the electrodes of the electrode pair being exposed to ambient environment.

5. The end effector of claim 1, a first electrode of the electrode pair being configured to contact tissue, and a second electrode of the electrode pair being configured as a reference electrode to the first electrode.

6. The end effector of claim 1, in which a center of a first electrode of the electrode pair being substantially aligned with a center of a second electrode of the electrode pair.

7. The end effector of claim 1, the planar substrate further comprising:
a frame comprising a pair of longitudinally extending struts that extend along the longitudinal axis; and
a membrane extending over each strut of the pair of longitudinally extending struts, and between the pair of longitudinally extending struts.

8. The end effector of claim 7, the electrode affixed to the planar substrate of the electrode pair being affixed to the membrane and between the pair of longitudinally extending struts.

9. The end effector of claim 1, the end effector comprising an array of at least four electrodes on the first side of the end effector,
the array comprising an electrode of the electrode pair that is on the first side of the end effector, and
the electrodes of the array being coplanar with each other.

10. An end effector of a catheter, the end effector comprising:
a plurality of spines;
an interlaced substrate traversing across a first spine of the plurality of spines on a first side of the end effector and traversing across a second spine of the plurality of spines on a second side of the end effector; and
an electrode pair comprising an electrode affixed to a spine of the plurality of spines and an electrode affixed the interlaced substrate substantially opposite the electrode affixed to the spine.

11. The end effector of claim 10, the electrodes of the electrode pair being exposed to ambient environment.

12. The end effector of claim 10, a first electrode of the electrode pair being configured to contact tissue, and a second electrode of the electrode pair being configured as a reference electrode to the first electrode.

13. The end effector of claim 10, in which a center of a first electrode of the electrode pair being substantially aligned with a center of a second electrode of the electrode pair.

14. The end effector of claim 10, the plurality of spines being substantially coplanar.

15. The end effector of claim 10, the end effector comprising:
a loop member comprising two spines of the plurality of spines and a connecting member joining distal ends of the two spines.

16. The end effector of claim 10, the end effector comprising:
a first loop member comprising a first pair of spines of the plurality of spines and a first connecting member joining distal ends of spines of the first pair of spines; and
a second loop member comprising a second pair of spines of the plurality of spines and a second connecting member joining distal ends of spines of the second pair of spines.

17. The end effector of claim 16, the interlaced substrate traversing each spine of the first pair of spines and the second pair of spines.

18. The end effector of claim 17, further comprising:
a first plurality of electrodes comprising a respective electrode affixed to each spine of the first pair of spines; and
a second plurality of electrodes affixed to the interlaced substrate and respectively opposite at least a portion of the first plurality of electrodes.

19. An end effector of a catheter, the end effector comprising:
a top side;
a bottom side opposite the top side;
a first substrate portion;
a second substrate portion;
an opening between the first and second substrate portions;
an interlaced substrate traversing on the top side of the end effector across the first substrate portion, extending through the opening, and traversing on a bottom side of the end effector across the second substrate portion; a first electrode pair comprising a bottom electrode affixed to the first substrate portion on the bottom side of the end effector and a top electrode affixed to the interlaced substrate on the top side of the end effector, the top electrode being disposed substantially opposite the bottom electrode of the first electrode pair;
a second electrode pair comprising a top electrode affixed to the second substrate portion on the top side of the end effector and a bottom electrode affixed to the interlaced substrate on the bottom side of the end effector, the bottom electrode being disposed substantially opposite the top electrode of the second electrode pair; and
a support frame configured to move the first substrate portion, the second substrate portion, and the interlaced substrate into a planar predetermined shape.

20. The end effector of claim 19, further comprising:
a contiguous substrate comprising the first substrate portion, the second substrate portion, and a nitinol support frame extending through the first substrate portion and/or the second substrate portion, the nitinol support frame comprising longitudinally extending struts, and the first substrate portion and/or the second substrate portion being disposed between the longitudinally extending struts;
a first membrane facing the top side of the end effector such that a portion of the first membrane is exposed to ambient environment and a portion of the first membrane is traversed by the interlaced substrate; and
a second membrane facing the bottom side of the end effector such that a portion of the second membrane is exposed to ambient environment and a portion of the second membrane is traversed by the interlaced substrate.
